(19) United States
(12) Patent Application Publication
(10) Pub. No.: US 2010/0311969 A1
(43) Pub. Date: Dec. 9, 2010

(54) Process For Preparation of Paliperidone

(75) Inventors: Amit Anant Chavan, Kalwa (IN); Ashutosh Vijay Joshi, Dombivli (IN); Manjunath Narayan Bhanu, Pashan Pune (IN)

Correspondence Address: Florek & Endres PLLC, 1156 Avenue of the Americas, Suite 600, New York, NY, 10036, US

(73) Assignee: WATSON PHARMA PRIVATE LIMITED, Andheri (East), Mumbai (IN)

(21) Appl. No.: 12/864,711

(22) PCT Filed: Feb. 5, 2009
(86) PCT No.: PCT/IN09/00077

§ 371 Date: Jul. 27, 2010

(30) Foreign Application Priority Data
Feb. 5, 2008 (IN) 249/MUM/2008

(51) Int. Cl. C07D 471/04

(52) U.S. Cl. 544/282

(57) ABSTRACT

The present invention relates to a process for preparation and purification of 3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one, also known as paliperidone or 9-hydroxy risperidone. The invention also relates to the preparation of intermediates useful in the process.

### FIELD OF THE INVENTION

The present invention relates to a process for preparation of 3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one, also known as paliperidone or 9-hydroxy risperidone and intermediates useful in the process.

### BACKGROUND OF THE INVENTION

The synthesis of 3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one, also known as paliperidone (Formula I), is disclosed in European Patent No. EP 368388.

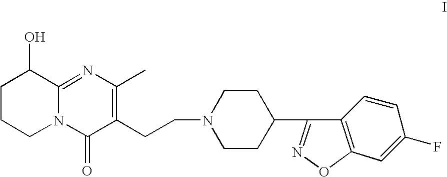

Paliperidone is an atypical antipsychotic drug developed by Janssen Pharmaceuticals. Chemically, paliperidone is a primary active metabolite of the antipsychotic drug risperidone. Paliperidone is approved by FDA for treatment of schizophrenia. It is also effective in the treatment of bipolar mania.

U.S. Pat. No. 5,688,799 discloses preparation of a precursor of paliperidone, namely, 3-(2-hydroxyethyl)-9-hydroxy-2-methyl-4H-pyrido[1,2-a]pyrimidin-4-one, by using 2-amino-3-pyridinol, 2-acetyl butyrolactone and p-toluene sulfonic acid.

US 20070260061 A1 pertains to preparation of a starting material of paliperidone, namely, crystalline 3-(2-hydroxyethyl)-9-hydroxy-2-methyl-4H-pyrido[1,2-a]pyrimidin-4-one, substantially free of 2-acetylbutyrolactone.

WO 2008024415 A2 discloses methods for preparing intermediates of paliperidone such as 3-benzyloxy-2-amino-pyridine (“BOPA”), 3-(2-hydroxyethyl)-6,7,8,9-tetrahydro-9-benzyloxy-2-methyl-4H-pyrrido[1,2-a]-pyrimidine-4-one (“HMBP”), 3-(2-chloroethyl)-2-methyl-9-benzyloxy-4H-pyrrido[1,2-a]-pyrimidine-4-one (“CMBP”), 3-(2-chloroethyl)-2-methyl-9-hydroxy-4H-pyrrido[1,2-a]-pyrimidine-4-one (“CMHP”), 3-(2-chloroethyl)-6,7,8,9-tetrahydro-9-hydroxy-2-methyl-4H-pyrrido[1,2-a]-pyrimidine-4-one (“CMHTP”). This patent also discloses preparation of 9-hydroxy risperidone (paliperidone) and reports the XRD pattern of CMHTP.

WO 2008021342 A2 discloses preparation of amorphous and crystalline forms of paliperidone. XRD patterns and solid state ¹³CNMR spectrum are also reported.

WO 2008021345 A2 relates to preparation of paliperidone from CMHTP in a variety of solvents under different reaction conditions.

WO 2008021346 A2 discloses a purification process to obtain paliperidone free of impurities.

WO 2008087557 A2 relates to preparation of intermediates of paliperidone such as 9-hydroxy-3-(2-chloroethyl)-2-methyl-4H-pyrrido[1,2-a]-pyrimidine-4-one and 3-(2-chloroethyl)-6,7,8,9-tetrahydro-9-hydroxy-2-methyl-4H-pyrrido[1,2-a]-pyrimidine-4-one.

EP 368388 B1 discloses preparation of paliperidone (formula I) by condensation of 3-(2-chloroethyl)-6,7,8,9-tetrahydro-9-hydroxy-2-methyl-4H-pyrido[1,2-a]pyrimidin-4-one (hereinafter referred to as “formula II” or “II”)

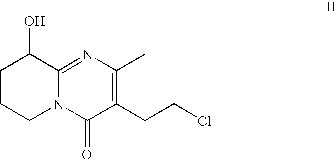

with 6-fluoro-3-(4-piperidinyl)-1,2-benzisoxazole.HCl (hereinafter referred to as “formula III” or “III”),

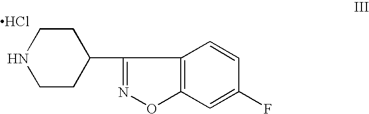

in the presence of an amine in methanol at 60° C., which is followed by its purification to obtain pure paliperidone. Pharmaceutical formulations containing paliperidone are also disclosed.

The intermediate of formula II in the synthesis of paliperidone can be obtained by hydrogenation of 3-(2-Chloroethyl)-2-methyl-9-(phenylmethoxy)-4H-pyrido[1,2-a]pyrimidin-4-one (hereinafter referred to as “formula IV” or “IV”):

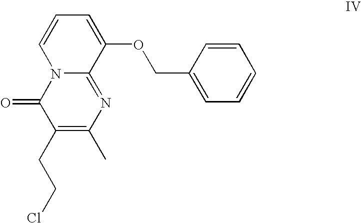

in methanol at normal pressure and at room temperature over Pd/C catalysts to obtain the oily residue of formula II.

EP 368388 B1 further discloses that the compound of formula II is condensed with the compound of formula III in the presence of an amine and methanol to obtain crude paliperidone (I). The crude paliperidone is purified by subjecting the crude paliperidone to two column chromatographic separations using a mixture of methanol and chloroform saturated with ammonia. The paliperidone obtained from the column chromatographic separations is further crystallized by using 2-propanone and finally recrystallized from 2-propanol.

There are a number of problems with the process described in EP 368388 B1. One problem is that the hydrogenation of the compound of formula IV as described in EP 368388 may produce dechlorinated product, i.e., ethyl tetrahydro pyridopyrimidine (hereinafter referred to as “formula V” or “V”), as a by-product. This may result in lower yield and inferior quality, which is undesirable.

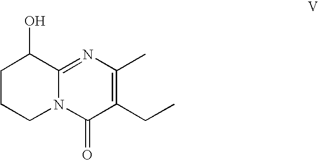

For example, when hydrogenation of the compound of formula IV was done in methanol as per EP 368388 B1, a significant quantity of undesired by-product of the compound of formula V was formed (more than 50% by HPLC) during this reaction.

An additional problem with the process described in EP 368388 B1 is the purification of crude paliperidone by column chromatography. This purification process is laborious, renders the process industrially undesirable and causes low yield.

### OBJECT OF THE INVENTION

The object of the present invention is to provide a simple and efficient process for the preparation of paliperidone.

Another object of the present invention is to provide a simple and efficient process for purifying paliperidone that avoids the use of column chromatography and/or eliminates the necessity of column chromatography for separation and/or purification.

A further object of the present invention is to provide a simple and efficient process for preparation of intermediates useful in the preparation of paliperidone.

An additional object of the present invention is to provide a process for preparation of 3-(2-Chloroethyl)-2-methyl-9-hydroxy-4H-pyrido[1,2-a]pyrimidin-4-one (formula VI) which can be used in the preparation of paliperidone.

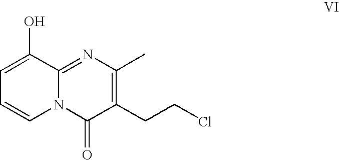

It is also an object of the present invention to provide a process for the preparation of a compound of formula II which can be used in the preparation of paliperidone wherein the process for preparation of the compound of formula II results in the production of small amounts of the compound of formula V, preferably less than 25% of the compound of formula V, more preferably less than 20% of the compound of formula V and most preferably less than 15% of the compound of formula V as determined by HPLC.

### SUMMARY OF THE INVENTION

The present invention relates to a process for preparation of 3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one, also referred to as paliperidone, 9-hydroxy risperidone or formula I comprising reacting the compound of formula II with the compound of formula III in inert solvents. The reaction is conducted in the presence of a base and at a suitable temperature that avoids and/or eliminates the use of column chromatography.

The present invention also relates to processes for the preparation of pure paliperidone, by means of simple purification techniques. As used herein, “pure paliperidone” refers to paliperidone that is at least 99.5% paliperidone, preferably at least 99.75% paliperidone and most preferably at least 99.8% paliperidone.

One embodiment of the invention comprises the preparation of the compound of formula II by hydrogenation of the compound of formulas IV or VI in the presence of a hydrogenation catalyst and hydrogen in an acidic medium. A further aspect of this embodiment produces the compound of formula II with less than 25% of the compound of formula V, preferably less than 20% of the compound of formula V and most preferably less than 15% of the compound of formula V as determined by HPLC.

Another embodiment of the invention comprises the preparation of 3-(2-Chloroethyl)-2-methyl-9-hydroxy-4H-pyrido[1,2-a]pyrimidin-4-one (formula VI) by reacting 3-benzyloxy-2-amino pyridine with 2-acetyl butyrolactone and phosphorus oxychloride in the presence of a solvent. This aspect of the invention may also include quenching of the reaction with water or a mixture of water and an organic solvent, adjusting the pH of the reaction and isolating of 3-(2-Chloroethyl)-2-methyl-9-hydroxy-4H-pyrido[1,2-a]pyrimidin-4-one (formula VI). A further aspect of this embodiment may include the step of extracting the compound of formula VI from the quenched reaction mass by use of a suitable extraction solvent such as methylene chloride prior to isolating the compound of formula VI. Alternatively, the compound for formula VI may be isolated without an extraction solvent by adding a suitable base to the quenched reaction mass. Once the compound of formula VI is isolated, it may be crystallized using an appropriate solvent system.

A further embodiment of the present invention is a process for the preparation of pure paliperidone comprising reacting the compound of formula II with the compound of formula III in inert solvents and in the presence of a base at a suitable temperature to obtain crude paliperidone. The crude paliperidone is purified into pure paliperidone by a process that does not require the use of column chromatography. This embodiment of the invention further comprises preparing the compound of formula II by hydrogenation of the compound of formula IV or VI in the presence of a hydrogenation catalyst and hydrogen in an acidic medium to produce the compound of formula II with less than 25% of the compound of formula V, preferably less than 20% of the compound of formula V and most preferably less than 15% of the compound of formula V as determined by HPLC. The compound of formula VI when used in this embodiment is prepared by reacting 3-benzyloxy-2-amino pyridine with 2-acetyl butyrolactone and phosphorus oxychloride in the presence of a solvent.

### DESCRIPTION OF THE DRAWINGS

FIG. 1 is a representative XRD pattern of the paliperidone prepared in accordance with the present invention.

### DETAILED DESCRIPTION OF THE INVENTION

The present invention relates to a process for preparation of 3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one, also referred to as paliperidone, 9-hydroxy risperidone or formula I comprising:
i) reacting the compound of formula II with the compound of formula III in inert solvents selected from the group consisting of alcohols, ketones, esters, ethers, hydrocarbons and mixtures thereof in the presence of a base at a suitable temperature;
ii) removing the solvent; and
iii) isolating the compound of formula I from solvents selected from the group consisting of water, alcohols, ketones, hydrocarbons or mixtures thereof.

Suitable solvents for the reaction described in step (i) include alcohols, ketones, esters, ethers, hydrocarbons and mixtures thereof. In one embodiment of the invention, the solvent is an alcohol or mixture of alcohols, preferably C₁ to C₄ alcohols such as methanol or IPA. In another embodiment of the invention, the solvent for the reaction in step (i) is ketones or mixture of ketones, preferably acetone.

In an alternate embodiment of the invention, the solvent for the reaction described in step (i) is a mixture of solvents selected from the group consisting of alcohols, ketones, esters, ethers and, hydrocarbons. Preferably, the mixture of solvents is a mixture of ketones and alcohols, most preferably a mixture of ketones and C₁ to C₄ alcohols, such as an acetone/methanol mixture. The ratio of ketone to alcohol is preferably about 1:9 to about 1:1, more preferably about 1:4 to about 1:2 and most preferably about 3:7.

The base employed in this process may be an organic base, an inorganic base or a mixture thereof. Examples of organic bases that may be used are tertiary amines such as triethylamine. Examples of inorganic bases that may be used are alkali metal or alkaline earth metal carbonates, bicarbonates or hydroxides, such as sodium carbonate.

The reaction temperature is preferably between 25-64° C. When an alcohol such as methanol is used as the solvent in step (i) the preferred reaction temperature is about 60° C. to about 63° C. When a mixture of solvents is used in step (i) the reaction is preferably conducted at reflux temperature. When a ketone such as acetone is used as the solvent in step (i) the reaction is preferably carried at reflux temperature.

The solvent can be removed by any means known in the art such as vacuum or distillation.

Isolating the compound of formula I in step (iii) may be performed with a solvent selected from water, alcohols, ketones and mixtures thereof. A preferred solvent is alcohols such as methanol, water or a mixture of a C₁ to C₄ alcohol and water, such as a methanol/water mixture. The ratio of alcohol to water used for the isolation is preferably between about 5:95 to about 50:50, most preferably between about 5:95 to about 65:35. In an alternate embodiment of the invention, isolating the compound of formula I in step (iii) may be performed with a solvent selected from water, alcohols, ketones and mixtures thereof. A preferred solvent is ketones such as acetone, water or a mixture of ketone and water, such as a acetone/water mixture. The ratio of ketone to water used for the isolation is preferably between about 5:95 to about 50:50, most preferably between about 5:95 to about 65:35.

If the compound of formula I does not exhibit sufficient purity after being isolated in step (iii), it may be further processed according to the present invention to increase the purity level.

The crude paliperidone obtained from step (iii) above or any other method, may be purified without the use of column chromatography by a purification process comprising:
a) reacting the crude paliperidone with an acid in water to form an aqueous reaction mixture;
b) extracting the aqueous reaction mixture with an organic solvent wherein the organic solvent is selected from the group consisting of esters, chlorinated solvents, hydrocarbons and mixtures thereof and creating an aqueous layer and an organic layer;
c) separating the aqueous layer and organic layer of step (b);
d) adjusting the pH of the aqueous layer with a base to a pH of about 8 to about 10;
e) extracting the pH adjusted aqueous layer with a chlorinated solvent;
f) separating the aqueous layer and the chlorinated solvent;
g) removing the chlorinated solvent to create a reaction mass; and
h) isolating the paliperidone from the reaction mass with a solvent selected from the group consisting of ketones, alcohols, water and mixtures thereof.

The acid employed in step (a) maybe an organic acid, a mineral acid or mixtures thereof. Preferably, the acid is an organic acid such as acetic acid.

The organic solvent employed in step (b) can be selected from the group consisting of esters, chlorinated solvents, hydrocarbons and mixtures. A preferred solvent is a chlorinated solvent such as methylene chloride.

The pH of the aqueous layer in step (d) may be adjusted with an organic base, an inorganic base or mixtures thereof. Examples of possible bases are described above. Some of the preferred bases that may be used include liquid ammonium or ammonium hydroxide. The pH of the aqueous layer should be adjusted to a pH of about 8 to about 10, and preferably a pH of about 8.5 to about 9.5.

The isolation of the paliperidone in step (h) is preferably performed with solvents selected from ketones, alcohols, water and mixtures thereof, more preferably C₁ to C₄ alcohols such as methanol, acetone, isopropyl alcohol, water and mixtures thereof.

One embodiment of the present invention also relates to a process for isolation of pure paliperidone from water with acid-base purification wherein the isolated paliperidone exhibits a purity of more than 99.5%. Isolation in water makes the process attractive industrially in terms of environmental friendliness and ease of operation.

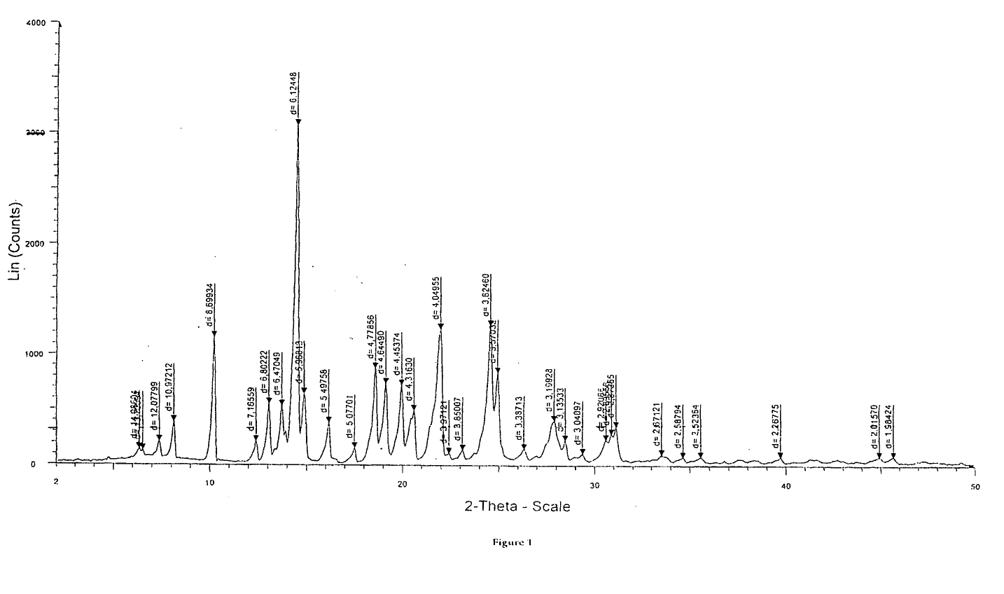

The paliperidone obtained in accordance with the present invention was subjected to recrystallization and precipitation in a variety of solvents and mixtures of solvents. The x-ray diffraction data revealed a nearly identical pattern regardless of the solvent or solvent system utilized. For example, samples of paliperidone obtained from Examples 13 and 14 below, as well as samples prepared by recrystallization/leaching of paliperidone in a variety of solvents such as acetone, isopropyl alcohol, ethyl acetate, DMF, methanol, acetonitrile, toluene, methanol/isopropyl ether, methanol/water, DMF/water and toluene/hexane exhibited nearly identical XRD patterns to the representative pattern shown in FIG. 1.

The present invention further relates to processes for the preparation of the compound of formula II comprising:
1) reacting 3-(2-Chloroethyl)-2-methyl-9-hydroxy-4H-pyrido[1,2-a]pyrimidin-4-one (formula VI) or 3-(2-Chloroethyl)-2-methyl-9-(phenylmethoxy)-4H-pyrido[1,2-a]pyrimidin-4-one (formula IV) [prepared as per EP 368388] with hydrogen and hydrogenation catalyst in an acidic medium to form 3-(2-chloroethyl)-6,7,8,9-tetrahydro-9-hydroxy-4H-pyrido[1,2-a]pyrimidin-4-one (formula II);
2) removing the acidic media from step (i) and
3) isolating the compound of formula II from a solvent selected from a group consisting of ketones, alcohols, water, hydrocarbons and mixtures thereof.

The catalyst used in step (1) is preferably Pd/C. Catalyst loading is 10-50% w/w of the wet catalyst, more preferably 10-20%. The hydrogen pressure applied during the reaction is in the range of 1-4 kg/cm², most preferably between 2-3 kg/cm². The reaction is performed at 25-60° C., more preferably at 30-40° C. The reaction medium used for the hydrogenation is selected from organic acids, aqueous mineral acids or mineral acids absorbed in alcoholic solvents. The preferred acids are organic acids such as acetic acid. The product is isolated in step (2) using a solvent selected from ketones, alcohols, water, hydrocarbons and mixtures thereof. Preferred solvents are a mixture of ketones and hydrocarbons such as acetone/hexane mixtures and/or water. When the product is isolated in step (2) using a mixture of ketones and hydrocarbons, the ratio of ketone to hydrocarbon is preferably about 10:90 to about 90:10, most preferably about 25:75 to about 50:50.

When the product is isolated in step (2) using water, the isolating step may further comprise adjusting the pH of the reaction mass with a base such as those previously described. The preferred base is an inorganic base such as sodium hydroxide. The pH of the reaction mass should be adjusted to about 4.5 to about 7, preferably about 5 to about 6.5, and most preferably about 5.5 to about 6. Isolation in water makes the process attractive industrially in terms of environmental friendliness and ease of operation.

3-(2-Chloroethyl)-2-methyl-9-hydroxy-4H-pyrido[1,2-a]pyrimidin-4-one, also referred to herein as the compound of formula VI and depicted below:

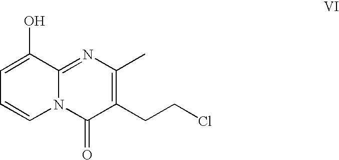

may be prepared in accordance with the present invention by a process comprising:
(1) reacting 2-amino-3-benzyloxy pyridine with 2-acetyl butyrolactone and POCl₃ in toluene at a suitable temperature to form a reaction mixture;
(2) quenching the reaction mixture by addition of water;
(3) adjusting the pH of the quenched reaction mixture with a base; and
(4) isolating compound of formula VI.

Preferably, the temperature of the reaction in step (1) is maintained between 50-110° C., more preferably 90-95° C.

The base added to the quenched reaction mixture in step (3) may be an organic base, an inorganic base or mixtures of the foregoing. Examples of possible inorganic bases include alkali metal or alkaline earth metal carbonates, bicarbonates or hydroxides such as ammonium hydroxide, sodium hydroxide, potassium hydroxide, sodium carbonate, sodium bicarbonate. Examples of possible organic bases include amines such as ammonium or tertiary amines such as triethylamine. The base should be added to the quenched reaction mixture in an amount that adjusts the pH to about 3.5 to about 7, preferably about 4 to about 5.

One embodiment for preparing the compound of formula VI may further comprise the use of an extraction solvent to assist in removing and isolating the compound of formula VI from the reaction mass. Preferably, the extraction solvent is an organic solvent or a mixture of water and organic solvent. A preferred organic solvent is chlorinated solvent such as methylene chloride. The extraction solvent may be added during the quenching step or subsequent to the quenching step. If an extraction solvent such as methylene chloride is employed in the process, the extraction solvent should be removed or substantially reduced after the addition of the base and prior to the isolation of the compound of formula VI. Once the extraction solvent has been removed or reduced, the compound of formula VI may be isolated by the addition of an alcohol. Exemplary alcohols for use in step (4) of this embodiment are C₁ to C₄ alcohols such as methanol, isopropyl alcohol or mixtures thereof.

In an alternative embodiment of the present invention, the compound of formula VI can be isolated from the quenched reaction (step (2)) without the addition of solvents for the extraction. In this alternative embodiment, the compound is isolated from the aqueous layer of the quenching step by neutralizing the aqueous layer with a base, preferably an inorganic base such as sodium hydroxide as described above. The compound isolated from aqueous layer may then be crystallized from a suitable solvent selected from alcohols, ketones, esters, ethers, hydrocarbons and mixtures thereof. Preferred solvents are alcohols, most preferably C₁ to C₄ alcohols such as methanol, isopropyl alcohol or mixtures thereof.

The following examples are intended to illustrate and not to limit the scope of the present invention.

### EXAMPLES

### Example 1

### Preparation of 3-(2-Chloroethyl)-2-methyl-9-hydroxy-4H-pyrido[1,2-a]pyrimidin-4-one (compound of formula VI)

35 ml of phosphorus oxychloride and 50 ml of toluene were charged in a reaction vessel. 50 g of 2-amino-3-benzyloxy pyridine was added to the above mixture at 25-30° C. The temperature of the reaction mass was raised to 50° C. and 48 g of 2-acetyl butyrolactone was added to the mass. The temperature of the mass was raised to 90-95° C. and maintained for 5 hours. 16 g of additional 2-acetyl butyrolactone was added to the reaction mass at 90-95° C. and the mixture was slowly stirred at 90-95° C. for an additional 1 hour to achieve the desired conversion (monitored by HPLC). 250 ml of water was then added to the reaction mass, which was stirred at 90° C. for 1 hour. It was then cooled to 25-30° C. Layers were separated. The toluene layer was further re-extracted with 50 ml of water. The combined aqueous layer was washed with 50 ml toluene. The layers were separated and 250 ml of methylene chloride was added to the aqueous layer and the pH of the solution was adjusted to 4.7-5.0 with 50% sodium hydroxide solution. The reaction mass was allowed to settle. Aqueous layer was extracted with 150 ml of methylene chloride. Both organic layers were combined and washed twice with 150 ml of water, then concentrated under vacuum. 50 ml of methanol was added to the mass and distilled under vacuum. Again, 50 ml of methanol was added and the reaction mass was cooled to 0-5° C. The solid mass was further stirred at 0-5° C. for 30 minutes. The mass was then filtered and washed twice with (2×50 ml) chilled methanol. Finally, the resultant solid was dried at 70° C. to obtain 28 g of formula VI.

Similarly, the compound of formula VI can be isolated from water by neutralizing the aqueous layer followed by its purification from methanol or isopropyl alcohol.

### Example 2

### Preparation of 3-(2-Chloroethyl)-6,7,8,9-tetrahydro-9-hydroxy-2-methyl-4H-pyrido[1,2-a]pyrimidin-4-one (Compound of formula II)

50 g of 3-(2-Chloroethyl)-2-methyl-9-hydroxy-4H-pyrido[1,2-a]pyrimidin-4-one (IV) was charged to a hydrogenation apparatus and 250 ml acetic acid was added to obtain a solution. 10 g of 10% Pd/C (wet) was charged to the solution. A hydrogen pressure of 3 kg/cm² was applied, and the mass was stirred at 25-30° C. for 4-5 hours until the reaction was complete. The reaction mass was filtered to remove Pd/C and then subjected to vacuum distillation at 65-70° C. to remove acetic acid. After distillation, an oily mass was obtained to which 150 ml of water and 250 ml of methylene chloride was added. The pH of the reaction mass was adjusted to 5.5-6.0 with 20% NaOH at 25-30° C. The reaction mass was stirred for 15-20 minutes at 25-30° C. The reaction mass was allowed to settle, and the layers were separated. The aqueous layer was extracted with 150 ml of methylene chloride. The organic layers were combined and washed with 250 ml of water. The combined organic layers were then subjected to vacuum distillation at 35° C. To the resulting oily mass was added 50 ml acetone, and the resulting solution was distilled atmospherically. Again, 50 ml acetone was added and the reaction mixture was heated to reflux for 15-20 minutes. While at reflux, 50 ml of hexane was added to the mass and the resulting mixture was then chilled to 0-5° C. The temperature was maintained for a further 30-45 minutes. The reaction mass was filtered, and the solid was washed twice with (2×25 ml) chilled hexane/acetone mixture. The resulting solid was dried under vacuum at 50° C. to obtain compound II. Dry wt.=30 g (compound of formula V, <10% by HPLC analysis, Purity of compound of formula II>85%).

### Example 3

The process of Example 2 was followed using compound (IV) instead of the compound of formula VI to obtain the compound of formula II (compound of formula V, <10% by HPLC analysis, Purity of compound of formula II>85%).

### Example 4

### Preparation of 3-(2-Chloroethyl)-6,7,8,9-tetrahydro-9-hydroxy-2-methyl-4H-pyrido[1,2-a]pyrimidin-4-one (Compound of formula II)

60 g of 3-(2-Chloroethyl)-2-methyl-9-hydroxy-4H-pyrido[1,2-a]pyrimidin-4-one (VI) was charged to a hydrogenation apparatus and 300 ml acetic acid was added to obtain a solution. 12 g of 10% Pd/C (wet) was charged to the solution. A hydrogen pressure of 3 kg/cm² was applied, and the mass was stirred at 25-30° C. for 4-5 hours until the reaction was complete. The reaction mass was filtered to remove Pd/C and then subjected to vacuum distillation below 60° C. to remove acetic acid. After distillation, an oily mass was obtained to which 180 ml of water was added. The reaction mixture was stirred for 30-45 minutes. The pH of the slurry was adjusted to 5.5-6.0 with 20% NaOH at 25-30° C. and stirred for 15-20 minutes at 25-30° C. The solid was filtered and washed twice with 60 ml water to obtain 36.5 g of compound of formula II.

### Example 5

### Preparation of Crude Paliperidone

79 g of 6-Fluoro-3,4-(piperidinyl)-1,2-benzisoxazole hydrochloride, 750 ml of methanol, and 78.2 g of triethyl amine were charged in a reaction vessel at 25-30° C. 75 g of the compound of formula II was added to the above mass. The reaction mixture was heated to 60-63° C. and then maintained at 60-63° C. to achieve desired conversion. The reaction mixture was then cooled to 40-45° C. Methanol was distilled off under reduced pressure to obtain a thick mass. 375 ml of methylene chloride was added to the reaction mass followed by 375 ml of water. The reaction mixture was stirred for 10-15 minutes and then filtered to obtain a clear solution. The layers were separated, and the aqueous layer was extracted twice with (2×190 ml) methylene chloride. Organic layers were combined and washed thrice with (3×190 ml) water. The organic layers were subjected to distillation under vacuum at 35° C. to remove methylene chloride. 75 ml of acetone was added to the thick mass and distilled to strip off methylene chloride. 750 ml of acetone was charged to the reaction mass, which was then heated to achieve reflux. The reflux was maintained for 30 minutes and then cooled to 0-5° C. and maintained for 45-60 minutes. The reaction mass was filtered, and the solid was washed twice with chilled (2×75 ml) acetone. The solid was dried at 70° C. to obtain crude paliperidone. Dry wt.=60 g (Purity of compound of formula I=99.07%).

### Example 6

### Preparation of Crude Paliperidone

79 g of 6-Fluoro-3-(4-piperidinyl)-1,2-benzisoxazole hydrochloride, 375 ml of methanol, and 78.2 g of triethyl amine were charged in a reaction vessel at 25-30° C. The reaction mixture was stirred for 5 minutes. 75 g of the compound of formula II and 375 ml methanol were added to the above mass. The reaction mixture was heated to 60-63° C. and then maintained at 60-63° C. to achieve the desired conversion. The reaction mixture was then cooled to 40-45° C. Methanol was distilled off under reduced pressure up to two volumes. 375 ml of water was added to the reaction mixture and stirred for 20-30 minutes at 25-30° C. The solid was filtered and washed twice with 150 ml water followed by 2×150 ml acetone to obtain 87 g crude paliperidone. (Purity of compound of formula I>97%).

### Example 7

### Preparation of Crude Paliperidone

5.3 g of 6-Fluoro-3-(4-piperidinyl)-1,2-benzisoxazole hydrochloride, 35 ml of methanol, and 5.2 g of triethyl amine were charged in a reaction vessel at 25-30° C. The reaction mixture was stirred for 5 minutes. 5 g of the compound of formula II and 15 ml acetone were added to the above mass. The reaction mixture was heated to reflux and then maintained at reflux temperature to achieve the desired conversion. The reaction mixture was then cooled to 40-45° C. The solvent was completely distilled off from the reaction mixture under reduced pressure. 10 ml of methanol was added to the reaction mixture and heated to 50-55° C. for 15-20 minutes. 25 ml water was added and the reaction mixture was cooled to 25-30° C. and maintained for 20-30 minutes. The solid was filtered and washed twice with 10 ml water followed by 2×10 ml acetone to obtain 6.3 g crude paliperidone. (Purity of compound of formula I>97%).

### Example 8

### Preparation of Pure Paliperidone

60 g of the crude paliperidone prepared in Example 5 and 900 ml of water were added to a reaction vessel. The pH of reaction mixture was adjusted to 3.5-4.5 with acetic acid at 25-30° C. The reaction mass was stirred for 15-20 minutes at 25-30° C. The reaction mixture was extracted with methylene chloride 180 ml. The layers were separated, and the organic layer was discarded. The aqueous layer was again extracted with 120 ml methylene chloride. Then, 600 ml of methylene chloride was added to the reaction mass, and the pH was adjusted to 9.0-9.5 with liquor ammonia at 25-30° C. The reaction mass was then stirred for 15-20 minutes. The layers were separated, and the aqueous layer was extracted with 120 ml of methylene chloride. The organic layers were combined and washed thrice with 180 ml water. The washed organic layer was treated with 15 g silica. The treated organic layer was subjected to atmospheric distillation to remove methylene chloride at 25-30° C. Acetone (2×60 ml) was added to the concentrated mass and distilled off atmospherically. Finally, 300 ml of acetone was added to the reaction mass. Acetone was distilled up to 150 ml at atmospheric pressure. The slurry was cooled to 0-5° C. and maintained for 45-60 minutes. The reaction mass was filtered and the solid was washed twice with (2×60 ml) chilled acetone. The solid was dried under vacuum at 80° C. to obtain pure paliperidone. Dry wt.=45 g (Purity of compound of formula I=99.81%).

### Example 9

### Preparation of Pure Paliperidone

14 g of crude paliperidone and 210 ml of water were added to a reaction vessel. The pH of reaction mixture was adjusted to 3.5-4.5 with acetic acid at 25-30° C. The reaction mass was stirred for 15-20 minutes at 25-30° C. The reaction mixture was extracted with methylene chloride 42 ml. The layers were separated, and the organic layer was discarded. The aqueous layer was again extracted with 28 ml methylene chloride. 140 ml of methylene chloride was added to the reaction mass, and the pH of the aqueous layer was adjusted to 9.0-9.5 with liquor ammonia at 25-30° C. The reaction mass was then stirred for 15-20 minutes. The layers were again separated, and the aqueous layer was extracted with 28 ml of methylene chloride. The organic layers were combined and washed thrice with 42 ml water. The washed organic layer was treated with 3.5 g silica. The treated organic layer was subjected to atmospheric distillation to remove methylene chloride at 25-30° C. Methanol (280 ml) was added to the concentrated mass and distilled off atmospherically. Acetone (70 ml) was added to the slurry and distilled off atmospherically. Acetone was distilled up to 35 ml at atmospheric pressure. The slurry was cooled to 0-5° C. and maintained for 45-60 minutes. The reaction mass was filtered and the solid was washed twice with (2×14 ml) chilled acetone. The solid was dried under vacuum at 80° C. to obtain pure paliperidone. Dry wt.=10 g.

### Example 10

### Preparation of Pure Paliperidone

15 g of the crude paliperidone and 75 ml of water were added to a reaction vessel. The pH of reaction mixture was adjusted to 3.5-4.5 with acetic acid at 25-30° C., and the reaction mass was stirred for 15-20 minutes at 25-30° C. The reaction mixture was extracted twice with methylene chloride (15 ml). The layers were separated, and the organic layer was discarded. The aqueous layer was treated with charcoal for 30 minutes. The pH of the aqueous layer was adjusted to 9.0-9.5 with liquor ammonia at 25-30° C. The reaction mass was stirred for 1 hour at 25-30° C. The solid was then filtered and washed twice with 30 ml of water followed with 15 ml acetone. The solid was transferred to a flask and refluxed with 75 ml acetone for 20-30 minutes. The reaction mixture was cooled to 25-30° C. and maintained for 30 minutes. The solid was filtered and washed twice with 15 ml acetone to obtain 11.5 g pure paliperidone.

Similarly, crude paliperidone prepared as in example 7 was purified as per the process followed in example 10 to obtain a pure paliperidone. (Purity of compound of formula I=99.86%).

### Example 11

### Preparation of Pure Paliperidone

14 g of crude paliperidone and 70 ml of water were added to a reaction vessel. The pH of the reaction mixture was adjusted to 3.5-4.5 with acetic acid at 25-30° C., and the reaction mass was stirred for 15-20 minutes at 25-30° C. The reaction mixture was extracted twice with methylene chloride (14 ml). The layers were separated and the organic layer was discarded. 140 ml of methylene chloride was added to the reaction mass, and the pH of the aqueous layer was adjusted to 9.0-9.5 with liquor ammonia at 25-30° C. The reaction mass was then stirred for 15-20 minutes. The layers were again separated, and the aqueous layer was extracted with 28 ml of methylene chloride. The organic layers were combined and washed thrice with 42 ml of water. The washed organic layer was treated with 3.5 g silica. The treated organic layer was subjected to atmospheric distillation to remove methylene chloride at 25-30° C. Isopropanol (280 ml) was added to the concentrated mass and distilled off atmospherically. Acetone (70 ml) was added to the slurry, and 35 ml of solvent was distilled off atmospherically. The slurry was cooled to 0-5° C. and maintained for 45-60 minutes. The reaction mass was filtered, and the solid was washed twice with (2×14 ml) chilled acetone. The solid was dried under vacuum at 80° C. to obtain pure paliperidone. Dry wt.=11 g.

### Example 12

Paliperidone (5 g) obtained from Example 10 was dissolved in isopropanol (400 ml) at reflux temperature. The clear mass was concentrated up to 4 volumes. Acetone (25 ml) was added to the slurry and 13 ml of solvent was distilled off atmospherically. The slurry was cooled to 0-5° C. and maintained for 45-60 minutes. The reaction mass was filtered, and the solid was washed twice with (2×5 ml) chilled acetone. The solid was dried under vacuum at 80° C. to obtain pure paliperidone. Dry wt.=4.5 g.

### Example 13

Paliperidone (2 g) was slurried in acetone (80 ml). The temperature was raised to 55-57° C. and maintained for 1 hour. The suspension was cooled to 25-30° C. The product was filtered and dried at 70° C.

### Example 14

Paliperidone (1.5 g) was dissolved in toluene (45 ml) at 90-95° C. Hexane (90 ml) was added to the solution. The suspension was cooled to 25-30° C. and stirred for 30 minutes. The product was filtered and dried at 70° C.

While certain preferred and alternative embodiments of the invention have been set forth for purposes of disclosing the invention, modifications to the disclosed embodiments may occur to those who are skilled in the art. Accordingly, this specification is intended to cover all embodiments of the invention and modifications thereof which do not depart from the spirit and scope of the invention.

What is claimed is:

1. A process for preparing 3-(2-Chloroethyl)-2-methyl-9-hydroxy-4H-pyrido[1,2-a]pyrimidin-4-one (compound of formula VI) comprising:
i) reacting 2-amino 3-benzyloxy pyridine with 2-acetyl butyrolactone and POCl₃ in toluene to form a reaction mixture;
ii) quenching the reaction mixture by adding water;
iii) adjusting the pH of the quenched reaction mixture with a base,
iv) isolating 3-(2-Chloroethyl)-2-methyl-9-hydroxy-4H-pyrido[1,2-a]pyrimidin-4-one.

2. The process of claim 1, wherein the base is sodium hydroxide.

3. The process of claim 1, wherein the base is an amine.

4. The process of claim 1, wherein the pH is adjusted to about 3.5 to about 7.

5. The process of claim 4, wherein the pH is adjusted to about 4 to about 5.

6. The process of claim 1 further comprising extracting the quenched reaction mass with an extraction solvent.

7. The process of claim 6 wherein the extraction solvent is methylene chloride.

8. The process of claim 6 wherein the 3-(2-Chloroethyl)-2-methyl-9-hydroxy-4H-pyrido[1,2-a]pyrimidin-4-one is isolated with an alcohol.

9. The process of claim 8 wherein the alcohol is methanol or isopropyl alcohol.

10. The process of claim 1 wherein the 3-(2-Chloroethyl)-2-methyl-9-hydroxy-4H-pyrido[1,2-a]pyrimidin-4-one is isolated from water.

11. The process of claim 1 further comprising the step of crystallizing 3-(2-Chloroethyl)-2-methyl-9-hydroxy-4H-pyrido[1,2-a]pyrimidin-4-one from an alcohol or a mixture of alcohols.

12. A process for preparing 3-(2-Chloroethyl)-6,7,8,9-tetrahydro-9-hydroxy-2-methyl-4H-pyrido[1,2-a]pyrimidin-4-one (compound of formula II) comprising:
i) reacting 3-(2-Chloroethyl)-2-methyl-9-(phenylmethoxy)-4H-pyrido[1,2-a]pyrimidin-4-one (compound of formula IV) or 3-(2-Chloroethyl)-2-methyl-9-hydroxy-4H-pyrido[1,2-a]pyrimidin-4-one (compound of formula VI) with hydrogen and a hydrogenation catalyst in an acidic medium to form 3-(2-chloroethyl)-6,7,8,9-tetrahydro-9-hydroxy-4H-pyrido[1,2-a]pyrimidin-4-one,
ii) removing the acidic media from step (i) and
iii) isolating 3-(2-chloroethyl)-6,7,8,9-tetrahydro-9-hydroxy-4H-pyrido[1,2-a]pyrimidin-4-one with a solvent selected from the group consisting of ketones, alcohols, water, hydrocarbons and mixtures thereof.

13. The process of claim 12, wherein the acidic medium is acetic acid.

14. The process of claim 12, wherein the hydrogenation catalysts is Pd/C.

15. The process of claim 12, wherein the isolation solvent is a mixture of acetone and hexane.

16. The process of claim 12 wherein the isolation solvent is water.

17. The process of claim 16 further comprising the step of adjusting the pH of the reaction mass to about 4.5 to about 7.

18. A process for preparing crude 3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one (compound of formula I) comprising:
i) reacting 3-(2-Chloroethyl)-6,7,8,9-tetrahydro-9-hydroxy-2-methyl-4H-pyrido[1,2-a]pyrimidin-4-one with 6-fluoro-3-(4-piperidinyl)-1,2-benzisoxazole.HCl in the presence of a base and an inert solvent selected from the group consisting of alcohols, ketones, esters, ethers, hydrocarbons and mixtures thereof;
ii) removing the solvent from step (i) and
iii) isolating crude paliperidone from a solvent selected from the group consisting of ketones, alcohols, water, hydrocarbons and mixtures thereof.

19. The process of claim 18, wherein the solvent of step (i) is an alcohol or ketone.

20. The process of claim 19 wherein the solvent is methanol or acetone.

21. The process of claim 18 wherein the solvent of step (i) is a mixture of a ketone and an alcohol.

22. The process of claim 21 wherein the solvent is a mixture of acetone and methanol.

23. The process of claim 18, wherein the base is an organic base.

24. The process of claim 23, wherein the base is triethylamine.

25. The process of claim 18, wherein the solvent in step (iii) is selected from the group consisting of water, C₁ to C₄ alcohol, ketone and mixtures thereof.

26. The process of claim 25, wherein the solvent is a mixture of water and C₁ to C₄ alcohol or water and acetone.

27. A process for purifying paliperidone comprising:
a) reacting crude paliperidone with an acid in water to form an aqueous reaction mixture;
b) extracting the aqueous reaction mixture with an organic solvent wherein the organic solvent is selected from the group consisting of esters, chlorinated solvents, hydrocarbons and mixtures thereof and creating an aqueous layer and an organic layer;
c) separating the aqueous layer and organic layer of step (b);
d) adjusting the pH of the aqueous layer with a base to a pH of about 8 to about 10;
e) extracting the pH adjusted aqueous layer with a chlorinated solvent;
f) separating the aqueous layer and the chlorinated solvent;
g) removing the chlorinated solvent to create a reaction mass; and
h) isolating the paliperidone from the reaction mass with a solvent selected from the group consisting of ketones, alcohols, water and mixtures thereof.

28. The process of claim 27, wherein the acid is acetic acid.

29. The process of claim 27, wherein the chlorinated solvent is methylene chloride.

30. The process of claim 27, wherein the base is ammonium hydroxide.

30. The process of claim 27, wherein the solvent in step (h) is selected from the group consisting of methanol, isopropyl alcohol, acetone and mixtures thereof.

31. The process of claim 27, wherein the solvent of step (h) is water.
